(19) United States
(12) Patent Application Publication
(10) Pub. No.: US 2021/0068662 A1
(43) Pub. Date: Mar. 11, 2021

(54) METHODS AND SYSTEMS FOR NEAR INFRARED SPECTROSCOPY

(71) Applicants: Zeinab Barati, Fairbanks, AK (US); Kambiz Pourrezaei, Fairbanks, AK (US)

(72) Inventors: Zeinab Barati, Fairbanks, AK (US); Kambiz Pourrezaei, Fairbanks, AK (US)

(73) Assignee: UNIVERSITY OF ALASKA FAIRBANKS, Fairbanks, AK (US)

(21) Appl. No.: 16/960,274

(22) PCT Filed: Apr. 2, 2019
(86) PCT No.: PCT/US2019/025357

§ 371 Date: Jul. 6, 2020

Related U.S. Application Data
(60) Provisional application No. 62/651,558, filed on Apr. 2, 2018.

(51) Int. Cl. A61B 5/00; A61B 5/0478; G01N 21/359

(52) U.S. Cl. CPC A61B 5/0075; A61B 5/0006; A61B 2503/40; A61B 5/4064; G01N 21/359; A61B 5/0478

(57) ABSTRACT

Methods and systems are disclosed for remotely and/or automatically controlling a probe to measure signals.

### CROSS REFERENCE TO RELATED PATENT APPLICATION

This application claim priority to U.S. Provisional Application No. 62/651,558 filed Apr. 2, 2018, which is herein incorporated by reference in its entirety.

### BACKGROUND

Long-term recording of cerebral oxygenation and hemodynamic activity is desired to assist in the study of ischemic stroke, epilepsy, and other neurological disorders. Typically, animal testing is done to ensure the safety of humans, but producing consistent results using animals can be difficult to accomplish due to the small size of the animals, as well as the animal needing freedom of movement for accurate results. Further, brain injuries (e.g., infarcts) in animals evolve over time and can take days to months to fully develop.

One method of monitoring perfusion is Laser Doppler Flowmetry (LDF). LDF provides an estimate of perfusion in monitored tissue. However, LDF has several limitations including high sensitivity to movement, and high signal variability. Further, bone (e.g., the skull of the animal) needs to be removed or thinned for accurate LDF readings of the brain. Thus, LDF has limitations in obtaining a secure and prolonged attachment to an animal, as well as consistent measurements over a period of time.

Additionally, Electroencephalography (EEG) is used to monitor and record electrical activity of the brain while studying animals. Current EEG methods require animals to be anesthetized or restrained in order to achieve relatively long and stable measurements. However, doing so limits the range of natural behaviors of the animals, which prevents obtaining accurate results. Thus, much like LDF, EEG has limitations in obtaining a secure and prolonged attachment to an animal while allowing the animal to move freely.

### SUMMARY

It is to be understood that both the following general description and the following detailed description are exemplary and explanatory only and are not restrictive, as claimed. Provided are methods and systems for near infrared spectroscopy.

In one embodiment, an apparatus comprises a probe having a plurality of light sources and photodetectors. The light sources may be located a first distance and a second distance away from the photodetectors. The light sources emit light and the photodetectors detect the light scattered within a living organism. The apparatus can also comprise a controller in communication with the probe. The controller can be configured to receiving a signal from a computing device to initiate a scan. The controller can sequentially activate each of the light sources to emit light in response to receiving the signal to initiate the scan. The controller can receive a measurement from the photodetectors that represents the detected light scattered in the living organism. The controller can transmit the measurement to the computing device.

In another embodiment, a method may comprise receiving a signal to initiate a scan from a computing device. The method further comprises sequentially activating a plurality of light sources to emit light in response to receiving the signal to initiate the scan. The light sources may be located a first distance and a second distance away from a plurality of photodetectors. The method also comprises receiving, from the plurality of photodetectors, a measurement that represents detected light scattered in a living organism. The measurement may be transmitted to the computing device.

In a further embodiment, a method comprises wirelessly transmitting, from a computing device to a Near Infrared Spectroscopy (NIRS) apparatus, a signal to initiate a scan. In response to the signal to initiate the scan, the NIRS apparatus can sequentially activate a plurality of light sources to emit infrared light. The light sources may be located a first distance and a second distance away from a plurality of photodetectors. Based on the activation of the light sources, a measurement may be received from the plurality of photodetectors. The measurement may represent the detected infrared light scattered in a living organism. The measurement can be transmitted from the NIRS apparatus to the computing device. Perfusion and oxygenation information for the living organism can be generated based on the measurement.

Additional advantages will be set forth in part in the description which follows or may be learned by practice. The advantages will be realized and attained by means of the elements and combinations particularly pointed out in the appended claims.

### BRIEF DESCRIPTION OF THE DRAWINGS

The accompanying drawings, which are incorporated in and constitute a part of this specification, illustrate embodiments and together with the description, serve to explain the principles of the methods and systems:

FIG. 1 is a diagram illustrating an exemplary system;

FIG. 2 is a block diagram illustrating an exemplary measuring system;

FIG. 3 is a diagram illustrating an exemplary system;

FIGS. 4A-4B are diagrams illustrating exemplary systems;

FIGS. 5A-5C are diagrams illustrating exemplary systems;

FIG. 6 is a flowchart illustrating an exemplary method;

FIG. 7 is a flowchart illustrating an exemplary method; and

FIG. 8 is a block diagram illustrating an exemplary computing system.

### DETAILED DESCRIPTION

Before the present methods and systems are disclosed and described, it is to be understood that the methods and systems are not limited to specific methods, specific components, or to particular implementations. It is also to be understood that the terminology used herein is for the purpose of describing particular embodiments only and is not intended to be limiting.

As used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other components, integers or steps. “Exemplary” means “an example of” and is not intended to convey an indication of a preferred or ideal embodiment. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

The present methods and systems may be understood more readily by reference to the following detailed description of preferred embodiments and the examples included therein and to the Figures and their previous and following description.

As will be appreciated by one skilled in the art, the methods and systems may take the form of an entirely hardware embodiment, an entirely software embodiment, or an embodiment combining software and hardware aspects. Furthermore, the methods and systems may take the form of a computer program product on a computer-readable storage medium having computer-readable program instructions (e.g., computer software) embodied in the storage medium. More particularly, the present methods and systems may take the form of web-implemented computer software. Any suitable computer-readable storage medium may be utilized including hard disks, CD-ROMs, optical storage devices, or magnetic storage devices.

Embodiments of the methods and systems are described below with reference to block diagrams and flowchart illustrations of methods, systems, apparatuses and computer program products. It will be understood that each block of the block diagrams and flowchart illustrations, and combinations of blocks in the block diagrams and flowchart illustrations, respectively, can be implemented by computer program instructions. These computer program instructions may be loaded onto a general purpose computer, special purpose computer, or other programmable data processing apparatus to produce a machine, such that the instructions which execute on the computer or other programmable data processing apparatus create a means for implementing the functions specified in the flowchart block or blocks.

These computer program instructions may also be stored in a computer-readable memory that can direct a computer or other programmable data processing apparatus to function in a particular manner, such that the instructions stored in the computer-readable memory produce an article of manufacture including computer-readable instructions for implementing the function specified in the flowchart block or blocks. The computer program instructions may also be loaded onto a computer or other programmable data processing apparatus to cause a series of operational steps to be performed on the computer or other programmable apparatus to produce a computer-implemented process such that the instructions that execute on the computer or other programmable apparatus provide steps for implementing the functions specified in the flowchart block or blocks.

Accordingly, blocks of the block diagrams and flowchart illustrations support combinations of means for performing the specified functions, combinations of steps for performing the specified functions and program instruction means for performing the specified functions. It will also be understood that each block of the block diagrams and flowchart illustrations, and combinations of blocks in the block diagrams and flowchart illustrations, can be implemented by special purpose hardware-based computer systems that perform the specified functions or steps, or combinations of special purpose hardware and computer instructions.

Regional cerebral blood flow and electroencephalography (EEG) recordings are often performed in anesthetized animals to achieve relatively long stable measurements, but anesthetizing animals limits the range of natural behaviors that neuroscientists can study. Restraining mechanisms using helmets, hammocks, jackets or wraps are sometimes used to achieve long-term recordings in so called “freely moving” animals. Although these configurations enable experimentation in awake animals, these configurations are uncomfortable for the animals or fail if not tightened, restrict the range of voluntary movements, introduce stress, and require habituation to the restricted condition.

Multimodal brain recording is a key tool for gaining a comprehensive understanding of brain activity since any single imaging method is limited to observing a single aspect of brain function. However, simultaneous observations by separate modalities require overcoming various practical challenges such as instrument interferences, limited space to accommodate multiple sensors for different types of recordings, and increased cost. Moreover, repeated observations across modalities introduce inter-event signal variability bias due to environmental and physiological changes or learning effects. Various combinations of imaging methods proved to be useful depending on the research questions that are being asked. The combination of information about electrical activity of the brain with the corresponding hemodynamic changes which offers superior spatial information represents one of the most powerful examples of a multimodal imaging technique and is one that is capable of providing new insights into brain function. A hybrid imaging tool, as described herein, can be capable of recording hemodynamic activity as well as EEG, which will benefit not only epilepsy research but also, will enable answering numerous research questions in basic and cognitive neuroscience.

The present disclosure provides neuroscientists with a hybrid NIRS-EEG functional imaging tool for small animals for unprecedented investigations of neurovascular coupling in a number of neurological disorders including epilepsy and cerebral ischemia. In addition to the miniaturized NIRS modality, a wireless EEG module is described that allows noninvasive measurement of electrical activity concurrently with NIRS measurement or independently. A low cost, noninvasive, wireless EEG modality can be a desirable alternative to the existing subdural electrodes technique. Integration of such multimodal measurements of cortical activity will be a powerful means for neuroscience to reveal the interaction between electrophysiology (fast response) and hemodynamics (slow response) at high spatial and temporal resolution.

Typically, invasive techniques are used for recording EEGs in animals. For example, intracranial electrode implants, as well as intraperitoneal or subcutaneous implantable transmitters, are invasive, require technical surgical skills, and induce postoperative trauma and care that may confound results, increase stress, and increase the mortality rate of the animals.

The present disclosure describes in an exemplary embodiment a miniaturized wireless, LED-based NIRS for small animals and will adapt human EEG recording protocols to rodents, yielding a new technique which allows us to noninvasively record a faithful EEG signal from rat with a recording electrode placed at the surface of the scalp.

Epilepsy research will also benefit from NIRS for early detection of seizure onset and moreover, a telemetric EEG module is desirable for epilepsy studies where detecting spontaneous seizures in chronic models needs long term recording particularly for those seizures with no or minimal motor symptoms.

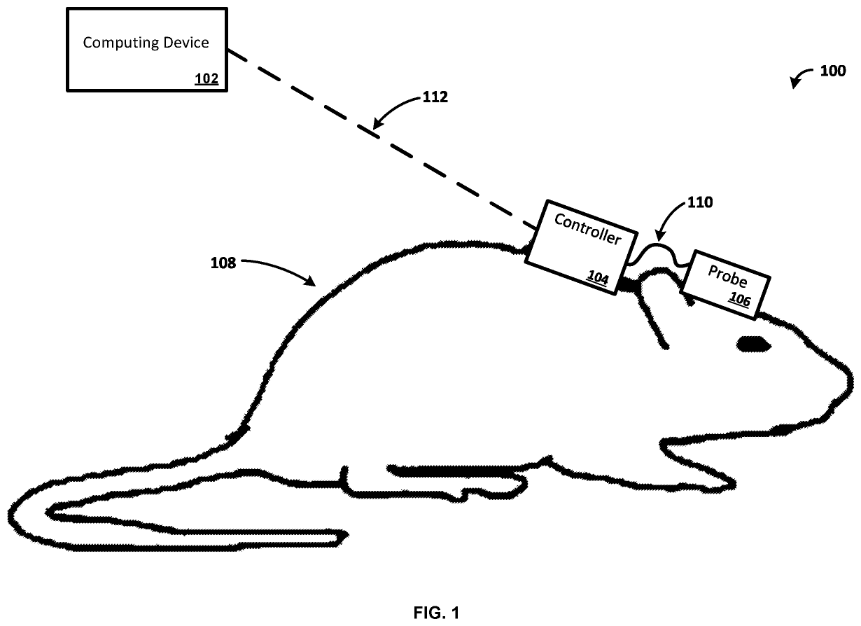

FIG. 1 illustrates a system 100 for remotely and/or automatically controlling a system for measuring signals. The system 100 can comprise one or more of a computing device 102 and/or a controller 104. In one exemplary embodiment, the controller 104 comprises a microcontroller. The system can further comprise one or more of a probe 106 in communication with the controller 104. Further, the probe 106 can also include a microcontroller (not shown) in communication with the controller 104. The controller 104 and the probe 106 can be located on an animal 108. The animal 108 can be a small rodent, such as a rat or a mouse, a cat, a dog, a primate, a human, and so forth. In one example, the probe 106 uses Near Infrared Spectroscopy (NIRS) for monitoring the oxygenation of tissue. In another example, the probe 106 monitors perfusion of tissue. The probe 106 can be configured to perform an Electroencephalography (EEG) scan of the animal 108. While an animal 108 is shown for ease of explanation, a person skilled in the art would appreciate that the system 100 can be configured to be used on any suitable organism such as a human, a primate, a dog, a cat, and the like.

The computing device 102 can be any type of electronic device. For example, the computing device 102 can be a computer, a smartphone, a laptop, a tablet, a wireless access point, a server, or any other electronic device. The computing device 102 can include an interface for communicating wirelessly using, for example, Wi-Fi, Bluetooth, cellular service, etc.

As shown, the controller 104 is communicatively coupled with the probe 106 via a communications connection 110. The controller 104 can use the communications connection 110 to provide control signals to the probe 106. For example, the communications connection 110 can directly couple the controller 104 and the probe 106 via one or more cables or wires (e.g., communications wires, Universal Serial Bus (USB), Ethernet, etc.). As another example, the communications connection 110 can be a wireless connection such that the controller 104 communicates wirelessly with the probe 106. The controller 104 can also use the communications connection 110 to provide power to the probe 106.

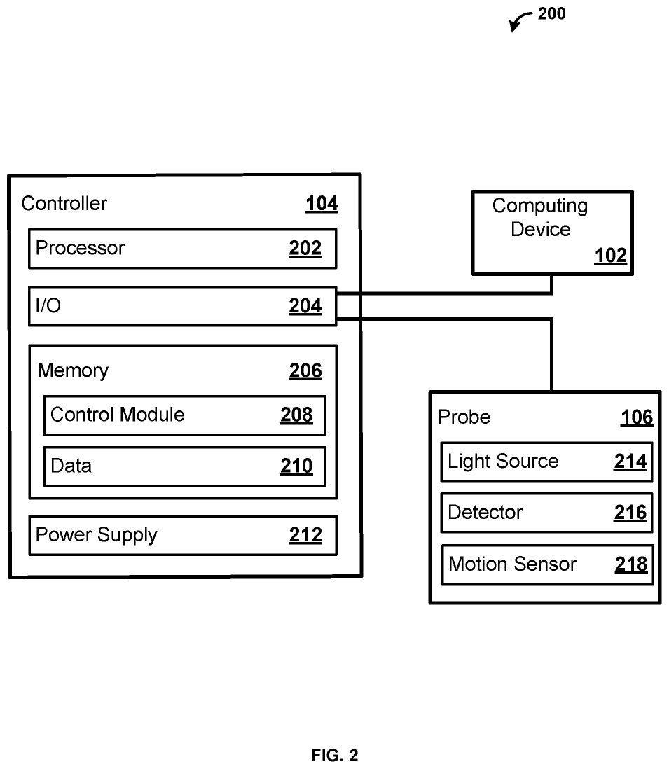

The controller 104 can include a processor, a memory, and an interface for communicating with other devices using wired connections or wirelessly using, for example, Wi-Fi, Bluetooth, cellular service as will be explained in more detail with regards to FIG. 2. In one example, the controller 104 controls the probe 106. The controller 104 can control the probe 106 based on data provided by sensors on the probe 106. For example, the controller 104 can receive data from the probe 106, and the controller 104 can use the data to determine how to control the probe 106. As another example, the controller 104 can receive data from the probe 106 and communicate the data to the computing device 102. As a further example, the controller 104 can perform an analysis on the data received from the probe 106. While a single controller 104 is illustrated for ease of explanation, a person skilled in the art would appreciate that any number of controllers may be present in the system 100. Further, while the controller 104 and probe 106 are illustrated as separate devices for ease of explanation, a person skilled in the art would appreciate that the controller 104 can include the functionality of the probe 106 and vice versa.

In one example, the controller 104 can be attached to the animal 108. For example, the controller 104 can be attached to the animal 108 using sutures. In another example, the controller 104 is attached to the animal 108 via adhesive (e.g., glue, tape). While several examples of methods to attach the controller 104 to the animal 108 are provided for ease of explanation, a person skilled in the art would appreciate that the controller 104 can be secured to the animal 108 via any suitable method. Alternatively, the controller 104 may not be attached to the animal 108. For example, the controller 104 can be attached to a holding device for the animal while the probe 106 is attached to the animal 108.

The probe 106 can be any suitable probe for measuring health related data of the animal 108. For example, the probe 106 can be capable of measuring the oxygenation of tissue and/or perfusion of blood through the tissue. As another example, the probe 106 can be configured to perform an Electroencephalography (EEG) scan of the animal 108. In one example, the probe 106 is made from a flexible material that allows for the animal 108 to move freely. For example, the flexible material can be a flexible film. In one example, the probe 106 is attached to the animal 108 using sutures. In another example, the probe 106 is attached to the animal 108 via adhesive (e.g., glue, tape). While several examples of methods to attach the probe 106 to the animal 108 are provided for ease of explanation, a person skilled in the art would appreciate that the probe 106 can be secured to the animal 108 via any suitable method. For example, the probe 106 and/or the controller 104 can be placed under the skin of the animal 108 via surgery. The probe 106 can include any sensors or sources for measuring signals of the animal 108. In one example, the probe 106 includes a light source and a detector as described in more detail with regards to FIG. 2.

As shown, the controller 104 and the probe 106 are attached to the animal 108 in such a manner that the animal 108 is not restrained. For example, the animal 108 is capable of moving freely while the controller 104 and the probe 106 are attached to the animal. In one example, the controller 104 and the probe 106 are self-sufficient (e.g., self-power, automated, etc.) devices that can allow the animal 108 to move freely. In this manner, the controller 104 and probe 106 are capable of providing data over an extended period of time without confining the movements of the animal 108. For example, the controller 104 and the probe 106 can enable continuous recording of cerebral oxygenation parameters which allows new fields of stroke research such as spatio-temporal study of stroke pathophysiology, peri-infarct depolarization, cerebral blood flow (CBF) monitoring, estimation of the hypoxic state of brain cells, confirmation of occlusion and reperfusion as well as identification of infarct formation and other pathophysiology in hemodynamically compromised brain regions.

As illustrated in FIG. 1, the computing device 102 and the controller 104 can be communicatively coupled via a communications connection 112. As an example, the computing device 102 and the controller 104 can communicate via a wireless network (e.g., Wi-Fi, Bluetooth). The computing device 102 and the controller 104 can exchange data using the communications connection 112. As an example, the controller 104 can provide data from the probe 106 to the computing device 102. The controller 104 can also provide the current operational status of the probe 106. For example, the controller 104 can provide data indicating that a sensor on the probe 106 is not functioning properly. As another example, the controller 104 can provide data relating to the last time a scan was performed using the probe 106. While the computing device 102 and the controller 104 are illustrated as directly communicating via the communications connection 112, a person skilled in the art would appreciate that the computing device 102 and the controller 104 can communicate via additional devices. For example, the computing device 102 can communicate with a device such as a server or wireless router, which in turn communicates with the controller 104.

The computing device 102 can also transmit settings or instructions to the controller 104 to manage operation of the controller 104. For example, the computing device 102 can provide software to the controller 104 that provides instruction for data collection from the probe 106. As another example, the computing device 102 can transmit settings to the controller 104 that indicate power management settings for the controller 104. As further example, the computing device 102 can transmit settings to the controller 104 that indicate when the controller 104 should provide data to the computing device 102. As one example, the computing device 102 can indicate start and stop times that the controller 104 should scan using the probe 106. As another example, the computing device 102 can indicate times that the controller 104 should start dynamically controlling the probe 106. In one example, a user of the computing device 102 actively selects the instructions or settings that are transmitted to the controller 104. In another example, the computing device 102 dynamically decides the instructions or settings that are transmitted to the controller 104 without input from a user. In another example, the computing device 102 receives input from a user indicating the preferences and/or settings the user would like the computing device 102 to implement. The computing device 102 can then automatically transmit instructions to the controller 104 based on the user indicated preferences and/or settings.

The computing device 102 can also transmit settings or instructions to the controller 104 to manage how the controller 104 controls the probe 106. For example, the computing device 102 can transmit settings to the controller 104 that indicate the timing of how the controller 104 should activate one or more light sources and/or detectors of the probe 106 in order to measure signals. As one example, the computing device 102 can indicate start and stop times that the controller 104 should activate the light sources. As another example, the computing device 102 can indicate times that the controller 104 should start dynamically controlling the probe 106. As a further example, the computing device 102 can indicate how the controller 104 should provide data to the computing device 102 from the probe 106. In one example, a user of the computing device 102 actively selects the instructions or settings that are transmitted to the controller 104. In another example, the computing device 102 dynamically decides the instructions or settings that are transmitted to the controller 104 without input from a user. In another example, the computing device 102 receives input from a user indicating the preferences and/or settings the user would like the computing device 102 to implement. The computing device 102 can then automatically transmit instructions to the controller 104 based on the user indicated preferences and/or settings. In one example, the user of the computing device 102 selects specific settings for the probe 106.

As a further example, the computing device 102 can provide a control signal to the controller 104 in order to control operation of the probe 106. The control signal can include settings for the probe 106, data related to settings of the probe 106, instructions for the probe 106, and any information related to the control of the probe 106. As an example, the computing device 102 can transmit a control signal to the controller 104 to activate one or more of the elements (e.g., sensors, light sources) of the probe 106. For example, the computing device 102 sends a control signal to the controller 104 to initiate a scan using the probe 106. The scan can comprise sequentially activating elements of the probe 106 to measure a characteristic of the animal 108.

In one example, the computing device 102 is a personal computer that has an application which controls the functionality of the controller 104 and/or the probe 106. For example, the computing device 102 can have data analysis software which controls operation of the controller 104 and the probe 106 in order to produce the desired data. In this manner, the computing device 102 is capable of controlling the controller 104 and the probe 106.

As will be appreciate by one skilled in the art, the communications connections shown in FIG. 1 can be, but need not be, concurrent. For example, the communications connections for each of the individual communications connections 110 and 112 can be established at a first time and then later terminated. Further, a person skilled in the art that any number of computing devices 102, controllers 104, and probes 106 can be implemented in the system 100.

FIG. 2 shows an exemplary system 200. As shown, the system 200 comprises a computing device 102, a controller 104, and a probe 106. While the controller 104 and the probe 106 are illustrated as separate devices for ease of explanation, in one exemplary embodiment the controller 104 and the probe 106 are configured on a single device. For example, a Near Infrared Spectroscopy (NIRS) apparatus can comprise the controller 104 and the probe 106. Further, the NIRS apparatus can also include the computing device 102.

The controller 104 comprises a processor 202, an input output interface (I/O) 204, a memory 206, and a power supply 212. In some examples, the controller 104 can include additional parts such as global positioning system (GPS), motion detectors, and so forth. While a single processor 202 is shown for ease of explanation, a person skilled in the art would appreciate that the controller 104 can include any number of processors 202. Further, the controller 104 can comprise one or more microcontrollers.

The processor 202 can perform various tasks, such as retrieving information stored in the memory 206, and executing various software modules. For example, the processor 202 can execute the control module 208 that provides instructions and/or settings to the probe 106. As an example, the control module 208 can provide instructions and/or settings for a scan utilizing the probe 106. In one example, the processor 202 can be a microcontroller.

As shown, the controller 104 is communicatively coupled via the I/O 204 with the computing device 102 and the probe 106. The I/O 204 can include any type of suitable hardware for communication with devices. For example, the I/O 204 can include direct connection interfaces such as Ethernet and Universal Serial Bus (USB), as well as wireless communications, including but not limited to, Wi-Fi, Bluetooth, cellular, Radio Frequency (RF), and so forth. Further, the I/O 204 can include a multiplexer for amplification, filtering, and/or digitization of signals. For example, the multiplexer can amplify, filter, and digitize the signals provide by the detector 216. As an example, the multiplexer can receive the signals (e.g., the output) from the detector 216. The multiplexer can amplify the received signals (e.g., the received output). The multiplexer can filter the received signals. The multiplexer can filter the received signals before or after the received signals are amplified. The multiplexer can then digitize the filtered signals. In an embodiment, the digitized signals represent spectral information characterizing light that is scattered in a living organism. As will be appreciated by one skilled in the art, the multiplexer can amplify, filter, and/or digitize the signals in any order and the present disclosure should not be limited to the aforementioned examples.

As shown, the probe 106 comprises a light source 214 and a detector 216. The light source 214 and the detector 216 can be mounted on a flexible film. The light source 214 can be any suitable light source providing light across any spectrum of light. For example, the light source 214 can be a Light Emitting Diode (LED), a laser, an X-ray source, an Ultra Violet (UV) source, and so forth. The detector 216 can be any suitable device for measuring light from the light source 214. For example, the detector 216 can be a photodetector that produces signals based on light detected by the detector 216. In one example, the light source 214 is an LED producing light infrared region of the electromagnetic spectrum, and the detector 216 is a photodiode capable of detecting the infrared light produced by the LED. Light source 214 can produce light in the near infrared light spectrum. As will be appreciated by one skilled in the art, the light source 214 can produce a large spectrum of light, while the detector 216 only measures a subset of the spectrum of light. While a single light source 214 and a single detector 216 are shown for ease of explanation, a person skilled in the art would appreciate that the probe 106 can contain any suitable number of light sources 214 (e.g., 2, 4, 10, 20, etc.) and detectors 216 (e.g., 2, 4, 10, 20, etc.). In one example, the probe 106 has four light sources 214 and eight detectors 216. While not shown for ease of explanation, the probe 106 may further comprise a microcontroller. The microcontroller can be configured to control the light source 214 and the detector 216.

The probe 106 can also include a motion sensor 218. The motion sensor 218 can include an accelerometer, a gyroscope, a Global Positioning System (GPS) sensor, or any other sensor for detecting motion. For example, the motion sensor 218 can detect motion of an animal that the probe 106 is attached to. The motion sensor 218 can produce motion data based on the movement of the animal. The motion sensor 218 can provide the motion data to the controller 104. The controller 104 can store the motion data, as well as provide the motion data to the computing device 102. The controller 104 and/or the computing device 102 can utilize the motion data to make one or more determinations regarding the motion of the animal. The controller 104 and/or the computing device 102 can utilize the motion data to determine an activity level of the animal. For example, the controller 104 and/or the computing device 102 can monitor and store the activity level of the animal over time. As an example, the controller 104 and/or the computing device 102 can utilize the motion data to compare the activity of the animal to the measurement data received from the detector 216 to determine if the motion of the animal has an impact on the measurements of the detector 216.

The controller 104 and/or the computing device 102 can utilize the motion data of the motion sensor 218 to ensure that the motion of the animal does not impact the measurements received via the detector 216. For example, the motion of the animal can impact the light measurements received by the detector 216. As an example, the detector 106 can receive a signal of light, and determine a measurement based on the signal of light. However, the detected measurement of light may be different depending on if the animal is still versus if the animal is moving. That is, the movement of the animal can introduce artifacts into the light as measured by the detector 216. Thus, the motion data can be utilized to filter (e.g., remove) any artifacts that motion of the animal might have introduced into the light as measured by the detector 216. Therefore, the controller 104 and/or the computing device 102 can utilize the motion data to filter out any artifacts that may have been introduced into the measurement of light by the movement of the animal. Accordingly, the controller 104 and/or the computing device 102 can utilize the motion data to ensure that the light measured by the detector 216 is accurate regardless if the animal is still or moves during the time the measurement is obtained. In an exemplary embodiment, an autoregressive (AR) model is applied to the measurement received from the detector 216 based on the motion sensor 218 data to remove any artifacts that the motion of the animal may have caused in the measurement.

The memory 206 includes a control module 208 and data 210. The memory 206 typically comprises a variety of computer readable media. As an example, readable media can be any available media and comprises, for example and not meant to be limiting, both volatile and non-volatile media, removable and non-removable media. The memory 206 can comprise computer readable media in the form of volatile memory, such as random access memory (RAM), and/or non-volatile memory, such as read only memory (ROM).

In another example, the memory 206 can also comprise other removable/non-removable, volatile/non-volatile computer storage media. The memory 206 can provide non-volatile storage of computer code, computer readable instructions, data structures, program modules, and other data for the controller 104. For example, a mass storage device can be a hard disk, a removable magnetic disk, a removable optical disk, magnetic cassettes or other magnetic storage devices, flash memory cards, CD-ROM, digital versatile disks (DVD) or other optical storage, random access memories (RAM), read only memories (ROM), electrically erasable programmable read-only memory (EEPROM), and the like.

The memory 206 can store software that is executable by the processor 202, including operating systems, applications, and related software. The memory 206 also includes data 210. The data 210 can include data received from the detector 216, settings or preferences for the light source 214, or any suitable type of data. As an example, the data 210 can include data related to the output of the light source 214 and the signals output by the detector 216. As another example, the data 210 can include data derived from the signals output by the detector 216. While not shown, a person skilled in the art would appreciate that the memory 206 can also include additional software and/or firmware for operating the controller 104.

The controller 104 also includes a power supply 212. The power supply 212 can be any suitable method of providing power to the controller 104 and the probe 106. For example, the power supply 212 can include a battery (e.g., Lithium-Ion, alkaline, etc.), a direct power connection (e.g., wired) to an external source (e.g., 120 V, 240 V), and/or a wireless power connection (e.g., induction) to an external source. The power supply 212 can comprise a voltage regulator configured to provide a constant voltage to the controller 104, as well as to the probe 106. The power supply 212 can also have a stable current source to provide stable current to the controller 104, as well as to the probe 106. Thus, the power supply 212 can provide a constant voltage and a stable current to the light source 214 and the detector 216 of the probe 106. In one example, the power supply 212 is a battery providing sufficient power for the controller 104 to operate, as well as sufficient power to operate the probe 106. In this manner, the controller 104 and the probe 106 can be untethered from other electronic devices in order to allow freedom of movement to an animal the controller 104 and the probe 106 are attached to. Further, as will be appreciated by one skilled in the art, the power supply 212 can include additional elements such as amplifiers, filters, and so forth. While a single power supply 212 is illustrated for ease of explanation, a person skilled in the art would appreciate additional power supplies 212 may be present that may include similar or different power sources.

In one example, the control module 208 includes the functionality to operate the probe 106. For example, the control module 208 includes the functionality to communicate with the probe 106 and provide operational instructions and/or preferences to the probe 106. As an example, the control module 208 can provide control signals to the probe 106 to run a scan. For example, the control module 208 can provide signals to the light source 214 to activate and produce light at a specific wavelength. As an example, the light source 214 may produce light in the 400-1000 nm range. For example, the light source 214 may produce light in the 600-700 nm, as well as light in the 800-900 nm range. Thus, the light source 214 can produce light at more than one wavelength. The different wavelengths of light may be produced simultaneously or at different times. While light in the 400-1000 nm range is used for ease of explanation, a person skilled in the art would appreciate that the light source 214 may produce light in any range and should not be limited to the aforementioned ranges.

As another example, the control module 208 can provide control signals to the probe 106 that controls the light source 214. For example, the control signals can dictate the light source 214 producing an output, the intensity of the output, how long the light source 214 should be activated, the wavelength of light produced by the light source 214, and so forth. The control module 208 can receive output signals and/or data from the detector 216, and the control module 208 can use the data to determine how the light source 214 should be controlled. For example, the control module 208 can recognize that the light source 214 is producing an output, but the detector 216 is not detecting any light. The control module 208 can determine that the light source 214 needs to increase the output in order for the detector 216 to detect the light. As another example, the control module 208 includes the functionality to run an analysis on the output of the detector 216. As another example, the control module 208 can receive input from a user that instructs the control module 208 to have the controller 104 activate the light source 214 and the detector 216 of the probe 106.

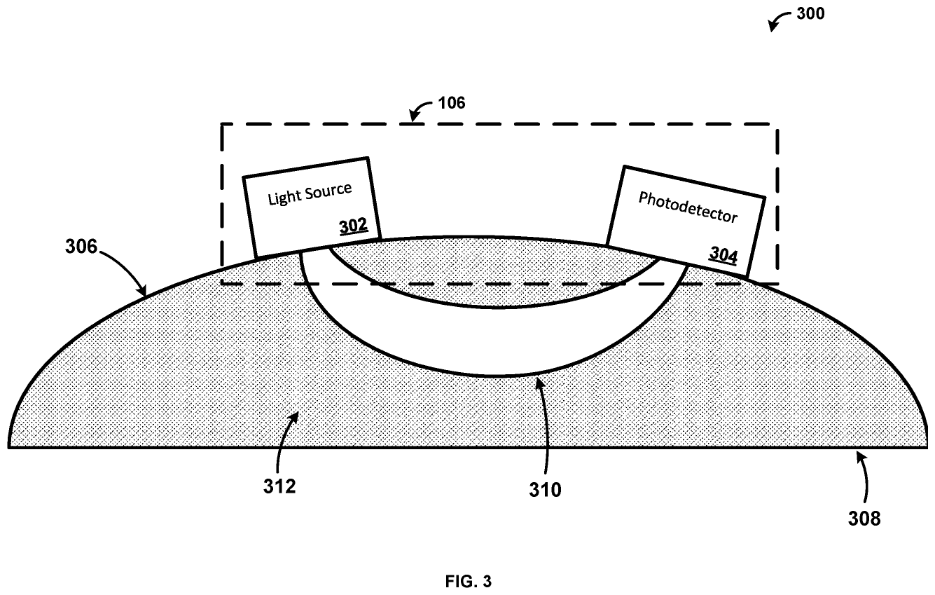

FIG. 3 shows an example of an operating environment 300 of the probe 106 including a light source 302 and a photodetector 304. While not shown for ease of explanation, the probe 106 can be configured to capture an EEG of the tissue 312. As shown, the light source 302 and the photodetector 304 are located on a surface 306 of a skull 308. The light source 302 is outputting a light 310 which travels through tissue 312 of the skull 308. The light 310 can be any suitable wavelength of light (e.g., UV, infrared, visible, X-ray). In one example, the light source 302 produces light in the infrared spectrum of light. The light source 302 can produce light in the near infrared spectrum of light. As an example, the light source 302 may produce light in the 400-1000 nm range. For example, the light source 302 may produce light in the 600-700 nm, as well as light in the 800-900 nm range. Thus, the light source 302 can produce light at more than one wavelength. The different wavelengths of light may be produced simultaneously or at different times. While light in the 400-1000 nm range is used for ease of explanation, a person skilled in the art would appreciate that the light source 302 may produce light in any range and should not be limited to the aforementioned ranges.

The depth of the light 310 penetration is a function of the distance between the light source 302 and the photodetector 304. The larger the distance between the light source 302 and the photodetector 312, the deeper the light 310 penetrates into the tissue 312. Thus, the distance between the light source 302 and the photodetector 304 can be varied in order to achieve varying penetration depths of the light 310 into the tissue 312. As shown, the surface 306 of the skull 308 is fully intact. In one example, the skull 308 does not need to be thinned or opened in order for the system 300 to function. In another example, the skin of the animal may be opened in order to attach the probe 106 directly to the surface 306 of the skull 308. Thus, the probe 106 may be placed underneath the skin of the animal.

As shown, the light 310 is output by the light source 302, enters through the surface 306 of the skull 308 and proceeds through the tissue 312. The photodetector 304 detects the light 310. In one example, the photodetector 304 detects the light 310 as the light 310 proceeds through the tissue 312 back towards the surface 306 of the skull 308. As another example, the photodetector 304 detects the light 310 after the light 310 exits the skull 308 and is detectable on the surface 306 of the skull 308. Thus, as shown, the light 310 passes a U-shaped pathway from the light source 302 to the photodetector 304. The light 310 is altered based on the tissue 312 within the skull 308 and indicates various aspects of the tissue 312, as well as hemodynamic activity related to the tissue 312. For example, the light 310 indicates the oxygenation of the blood, perfusion of blood within the tissue 312, whether an infarct is present, a volume of the infarct, the tissue around the infarct, and any normal tissue 312. The photodetector 312 outputs a signal to the controller 104 based on the received light 310. The output from the photodetectors 312 can represent spectral information characterizing the detected infrared light scattered within the tissue 312. Based on the change in the light 310 from the light source 302, data can be determined relating to the tissue 312, the perfusion of blood, and the oxygenation of the blood within the skull 308. For example, the output from the photodetector 304 can indicate the blood flow through the tissue 312 in order to monitor an infarct within the tissue 312. In one example, the output from the photodetector 304 can indicate the amount of oxygenation in the tissue 312. In this manner, the probe 106 is capable of measuring several characteristics related to the tissue 312, as well as hemodynamic activity of the tissue 312. While a skull is used for ease of explanation, a person skilled in the art would appreciate that the probe 106 may be placed on any part of the body and should not be limited to the aforementioned example.

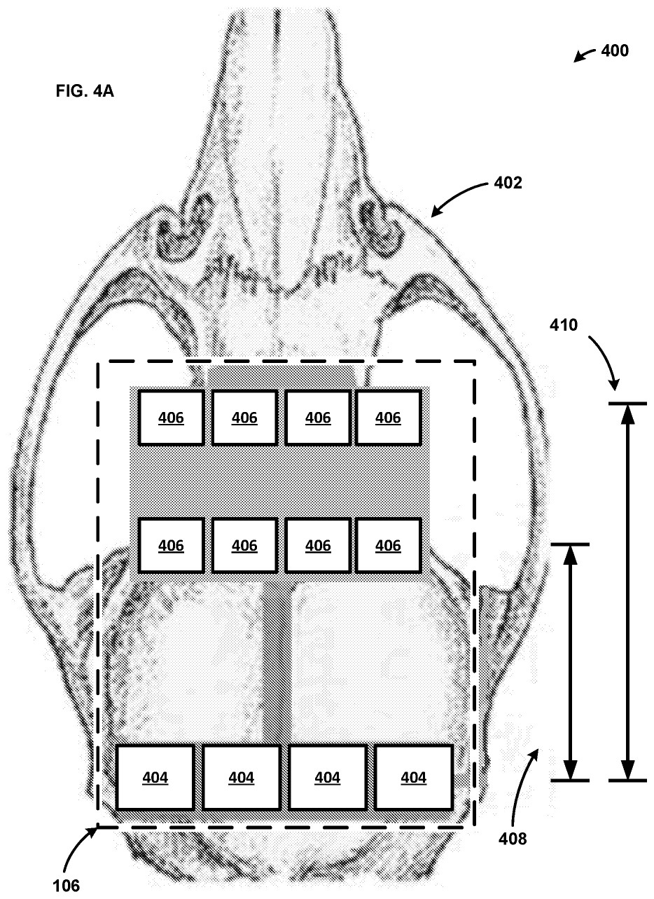

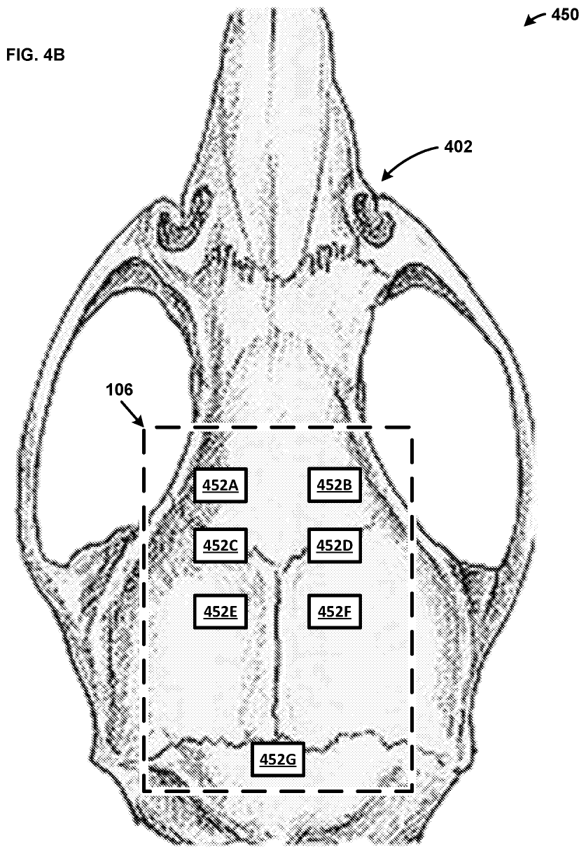

FIG. 4A shows an example system 400 including an implementation of the probe 106 on an animal skull 402. As shown, the probe 106 includes four light sources 404 and eight photodetectors 406. The lights sources 404 can be LEDs capable of emitting light in the infrared spectrum. As an example, the light sources 404 may produce light in the 400-1000 nm range. For example, the light sources 404 may produce light in the 600-700 nm, as well as light in the 800-900 nm range. Thus, the light sources 404 can produce light at more than one wavelength. The different wavelengths of light may be produced simultaneously or at different times. While light in the 400-1000 nm range is used for ease of explanation, a person skilled in the art would appreciate that the light sources 404 may produce light in any range and should not be limited to the aforementioned ranges.

The photodetectors 406 can be photodiodes that comprise six optical channels. The photodetectors 406 can be configured to monitor bilateral cortices of the brain. For example, the photodetectors 406 may monitor for signals from the bilateral motor and somatosensory cortices of the brain. Four of the photodetectors 406 are a first distance 408 from the light sources 404, and four of the photodetectors 406 are a second distance 410 from the light sources 404. In one example, the first distance 408 can be between 0-9 mm, and the second distance 410 can be between 10-20 mm. As another example, the first distance 408 is 8 mm, and the second distance 410 is 12 mm. As will be appreciated by on skilled in the art, the distances between the photodetectors 406 and the light sources 404 can vary depending on the size of the animal the probe is attached to and should not be limited to the aforementioned examples. For example, there may only be one set of photodetectors 406 at a single distance from the light sources 404. As another example, there may be any number of photodetectors at 406 at varying distances (e.g., 3, 5, 25, 50, 100, etc. different distances from the light sources 404). Further, additional light sources 404 may be present at a location that is different from the location of the light sources 404 of FIG. 4A. That is, a first set of light sources 404 may be a distance from a second set of light sources 404. Additionally, while four light sources 404 and eight photodetectors 406 are shown for ease of explanation, a person skilled in the art would appreciate the system 400 can comprise any number of light sources 404 and photodetectors 406.

As mentioned above, the penetration of the light through the skull 402 is a relative to the distance between the light source 404 and the photodetector 406. Thus, four of the photodetectors 406 detect light penetrating to a first depth within the skull 402, whereas four of the photodetectors 406 detect light penetrating to a second depth within the skull 402. As an example, the light detected by the photodetectors 406 the first distance 408 from the light sources 404 travels to a shorter depth within the skull 402, and thus travels a shorter pathway in comparison to the light detected by the photodetectors 406 the second distance 410 from the light sources 404. That is, the light detected by the photodetectors 406 the second distance 410 from the light sources 404 travels a deeper depth within the head/skull 402, and thus travels a longer pathway. Accordingly, the probe 106 is capable of measuring tissue at a variety of depths. Further, the position of the photodetectors 406 dictates the depth that the light penetrates within the skull 402.

In one example, the controller 104 calibrates the light sources 404 and the photodetectors 406. For example, the controller 104 can determine the output for each of the eight photodetectors 406 when all of the light sources 404 are inactive (e.g., turned off). The controller 104 can use this information to determine the background light and/or noise detected by the photodetectors 406 so that the background light and/or noise can be filtered out. As another example, the controller 104 can utilize the background light to calibrate the photodetectors 406 to improve the measurements of the photodetectors 406. The controller can also calibrate each of the photodetectors 406 individually because each photodetector 406 may receive different amounts of background light. While the controller 104 is described as calibrating the photodetectors 406 for ease of explanation, a person skilled in the art would appreciate that a computing device (e.g., the computing device 102 of FIGS. 1 & 2) could also calibrate the photodetectors 406.

In one example, the controller 104 controls the timing of light sources 404 of the probe 106 during a scan. As an example, the controller 104 activates the light sources 404 in a sequential manner. For example, the controller 104 activates one of the light sources 404 at a first frequency or wavelength of light. The eight photodetectors 406 each receive a corresponding signal based on the output from the light source 404. The eight photodetectors 406 then produce an output signal that is received by the controller 104. The controller 104 then activates one of the three remaining light sources 404 at the same frequency or wavelength of light. Again, the eight photodetectors 406 then produce an output signal that is captured by the controller 104. The controller 104 can continue to cycling through the light sources 404 in a round robin manner activating the light sources 404 at different frequencies or wavelengths of light. The controller 104 will continue to receive the outputs from the eight photodetectors 406 and store the data while proceeding through the scan. In an example, not all of the eight photodetectors 406 receive a light signal from each of the light sources 404. For example, six out of the eight photodetectors 406 can receive a light signal from one of the light sources 404 at a given frequency or wavelength. The two photodetectors 406 that do not receive the light signal may not receive the light signal due to the location of the light source 404 in relation to the two photodetectors, the anatomy of the skull 402, or any number of reasons as will be appreciated by one skilled in the art. The controller 104 can record which photodetectors 406 do not produce an output. That is, the controller 104 can record which photodetectors 406 do not receive the light signal. While describe the photodetectors 406 as not receiving the light signal is used for ease of explanation, a person skilled in the art would appreciate that the photodetectors 406 may receive trace amounts of the light signal.

The controller 104 can provide data related to the control of the light sources 404, as well as the data output by the photodetectors 406, to the computing device 102. In one example, the controller 104 provides the data to the computing device 102 after the scan is completed. In another example, the controller 104 provides the data to the computing device 102 at predetermined intervals of time. In a still further example, the controller 104 provides the data to the computing device 102 in real time as the controller 104 receives the data from the photodetectors 406. As will be appreciated by one skilled in the art, there are variety of ways and conditions to provide the data from the controller 104 to the computing device 102, and the disclosure should not be limited to the aforementioned examples.

FIG. 4B shows an example system 450 including another exemplary implementation of the probe 106 on the animal skull 402. While systems 400 and 450 are described in separate figures for ease of explanation, a person skilled in the art would appreciate that the probe 106 can include both systems in a single embodiment. That is, the probe 106 can include the light sources 404, the photodetectors 406, and the electrodes 452 in a single probe. As shown, the probe 106 includes seven electrodes 452A, 452B, 452C, 452D, 452E, 452F, and 452G. The electrodes 452 are placed on the animal skull 402 to monitor specific portions of the brain. For example, the electrode 452A is placed to monitor the right primary motor cortex, the electrode 452B is placed to monitor the left primary motor cortex, the electrode 452C is placed to monitor the right hind limb primary somatosensory cortex, the electrode 452D is placed to monitor the left hind limb primary somatosensory cortex, the electrode 452E is placed to monitor the right somatosensory cortex trunk region, the electrode 452F is placed to monitor the left somatosensory cortex trunk region, and the electrode 452G is a reference electrode (e.g., ground). The electrodes 452 can be utilized to perform an EEG of the brain within the animal skull 402. For example, the controller 104 can perform an EEG of the brain within the animal skull 402 via the probe 106. While the electrodes 452 are described as being placed to monitor specific portions of the brain within the animal skull 402, one skilled in the art would appreciate that the electrodes 452 may monitor any portion of the brain. Further, while six electrodes 452 are used for ease of explanation, a person skilled in the art would appreciate that the probe 106 may include any number of electrodes 452.

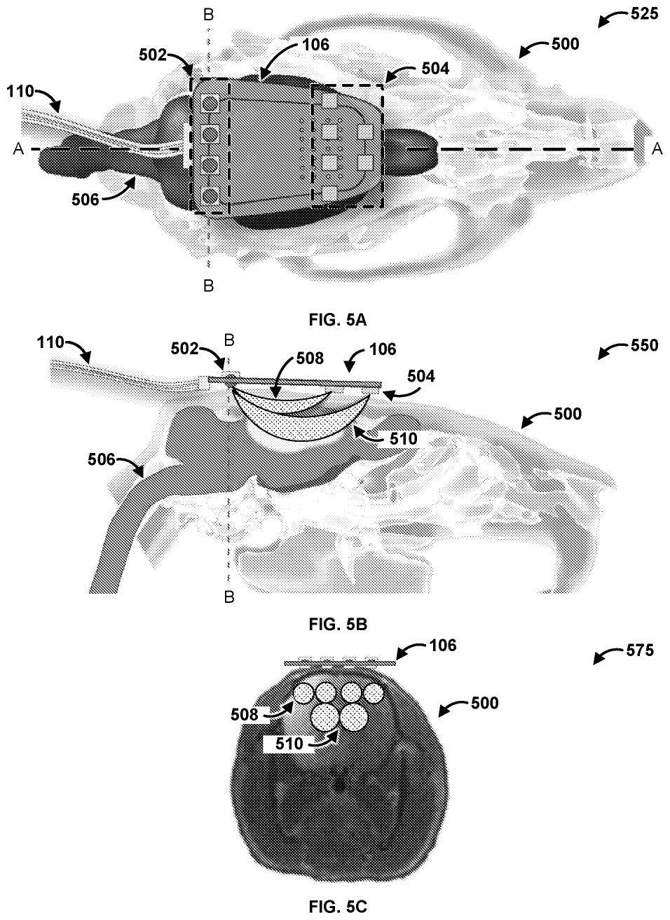

FIG. 5A is a diagram of an exemplary system 525. The system 525 has a first plane A-A and a second plane B-B. Specifically, FIG. 5A shows the probe 106 coupled to a skull 500 of an animal. In an exemplary embodiment, the skull 500 is of a rat. The probe 106 can be configured to determine characteristics of a brain 506 of the skull 500. As shown, the probe 106 has a communications connection 110 that can couple the probe with a controller (e.g., the controller 104 of FIGS. 1 & 2) and/or a computing device (e.g., the computing device 102 of FIGS. 1 & 2). The probe 106 has four light sources 502. The light sources 502 can be any suitable light source providing light across any spectrum of light. For example, the light sources 502 can be a Light Emitting Diode (LED), a laser, an X-ray source, an Ultra Violet (UV) source, and so forth. The light sources 502 can operate at the same wavelengths of light. The light sources 502 can operate at different wavelengths of light. The light sources 502 can be the same as the light sources 214 of FIG. 2, 302 of FIG. 3, and 404 of FIG. 4. The probe 106 also has si6 photodetectors 504. The photodetectors 504 can be the same as the photodetectors 216 of FIG. 2, 304 of FIG. 3, and 406 of FIG. 4. While six photodetectors 504 are shown for ease of explanation, a person skilled in the art would appreciate that the probe 106 can have any number of photodetectors 504.

FIG. 5B is a diagram of an exemplary system 550. FIG. 5B is a cross section of the system 525 of FIG. 5A along the A-A plane. As shown, the light sources 502 emit light that is detected by the photodetectors 504. The photodetectors 504 receive the light after the light traverses through the brain 506. The photodetectors 504 determine data based on the received light, and the photodetectors 504 provide the data to a computing device (e.g., the controller 104 and/or the computing device 102 of FIGS. 1 & 2) via the communications connection 110. Specifically, the light 508 travels a first depth and a first length from the light sources 502 that are located closer to the photodetectors 504. Stated differently, the light 508 travels along a short pathway through superficial tissue of the brain 506. In contrast, the light 510 travels a second depth and a second length from the light sources 502 that are located further away from the photodetectors 504. That is, the light 510 travels along a long pathway through deeper tissue of the brain 506. Accordingly, the probe 106 is capable of measuring two different depths into the brain 506 by utilizing two sets of photodetectors 504 that are located two different distances away from the light sources 502.

FIG. 5C is a diagram of an exemplary system 575. FIG. 5C is a cross section of the system 525 of FIG. 5A along the B-B plane. As shown, FIG. 5C indicates the path that the light 508 and the light 510 travels from each light source 502 to the photodetectors 504 though the skull 500. Specifically, each light source 502 has an associated path that the light travels from the light source 502 to the photodetectors 504 through the skull 500. Specifically, the photodetectors 504 that are located closer to the light sources 502 measure the light 508 that travels a shallower path into the skull 500. In contrast, the photodetectors 504 that are located further from the light sources 502 measure the light 510 that travels a deeper path into the skull 500. Thus, the placement of the photodetectors 504 and the light sources 502 directly impact the path that the light 508, 510 travels through the skull 500. Therefore, the position of the photodetectors 504 and the light sources 502 on the probe 106 can be modified in order to alter the path that the light 508, 510 travels through the skull 500. Stated differently, the path that the light 508, 510 travels through the skull can be manipulated and changed based on the location of the photodetectors 504 and the light sources 502 to modify the depth the light 508, 510 travels into the skull 500, as well as the distance the light 508, 510 travels. Accordingly, the probe 106 can be modified to be applicable to multiple beings such as other rodents, primates, dogs cats, humans, and so forth.

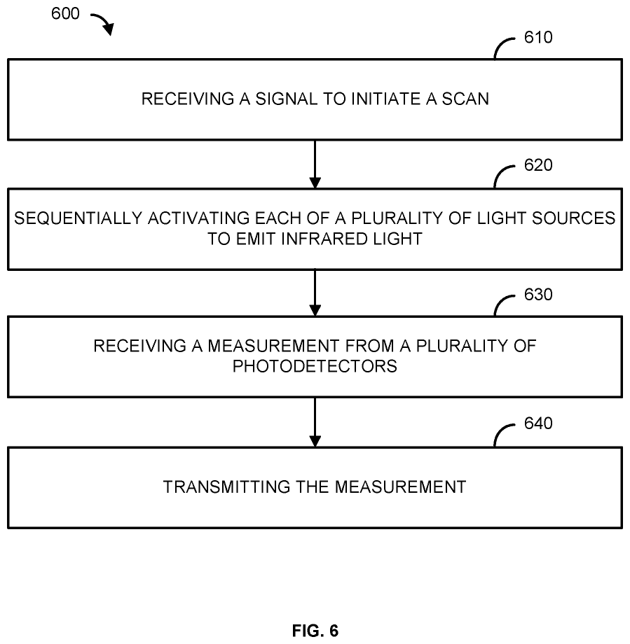

FIG. 6 is a flowchart of an example method 600. At step 610, a signal to initiate a scan is received. For example, a controller (e.g., the controller 104 of FIGS. 1 & 2) can receive a signal from a computing device (e.g., the computing device 102 of FIGS. 1 & 2) to initiate a scan. In one example, the signal to initiate the scan is received via a communications module (e.g., the communications link 112 of FIG. 1 and/or the I/O 204 of FIG. 2). In another example, the controller automatically initiates a scan based on settings and/or instructions previously sent by the computing device.

In step 620, a plurality of light sources can be sequentially activated to emit infrared light. The plurality of light sources can be associated with a probe (e.g., the probe 106 of FIGS. 1-5). For example, the controller can sequentially activate light sources (e.g., the light sources 214 of FIG. 2, 302 of FIG. 3, 404 of FIG. 4, and/or 504 of FIG. 5) to emit infrared light. The controller can automatically activate the light sources in response to receiving the signal to initiate a scan. The light sources can output the same wavelength of infrared light or different wavelengths of infrared light. The light sources can be positioned a first distance (e.g., the distance 408 of FIG. 4A) and a second distance (e.g., the distance 410 of FIG. 4A) from a plurality of photodetectors (e.g., the photodetectors 216 of FIG. 2, 304 of FIG. 3, 406 of FIG. 4, and/or 502 of FIG. 5). The light sources can be located on a skull (e.g., the skull 308 of FIG. 3, the animal skull 402 of FIG. 4, and/or the skull 500 of FIG. 5), and the light sources can output light into the tissue (e.g., the tissue 312 of FIG. 3 and/or the brain 506 of FIG. 5) within the skull. In one example, the light sources comprise LEDs.

As another example, in step 620 a plurality of electrodes can be activated to perform an EEG. For example, the controller can activate the electrodes (e.g., the electrodes 452 of FIG. 4B). The controller can automatically activate the electrodes in response to receiving the signal to initiate the scan. The electrodes can be located on a skull (e.g., the skull 308 of FIG. 3, the animal skull 402 of FIG. 4, and/or the skull 500 of FIG. 5), and the electrodes can monitor the tissue (e.g., the tissue 312 and/or the brain 506 of FIG. 5) within the skull. While activating the electrodes is described separately from activating the light sources, a person skilled in the art would appreciate that the plurality of light sources may be activated at the same time as the electrodes. That is, the controller may perform two scans concurrently. One scan using the light sources and photodetectors, and one scan using the electrodes. Further, the two different scans can be performed one after the other such that once the first scan is completed, the second scan automatically begins. However, the scans can also be performed at separate times.

In step 630, a measurement from a plurality of photodetectors is received. For example, the controller can receive the outputs from the photodetectors. The photodetectors can be associated with the probe (e.g., the probe 106 of FIGS. 1-5). The photodetectors can comprise photodiodes. The measurement can represent the detected infrared light (e.g., the light 310 of FIG. 3 and/or the light 508 of FIG. 5) scattered within a living organism (e.g., the animal 108 of FIG. 1). For example, the measurement can represent the detected light scattered within the tissue of a skull of the living organism (e.g., a brain of the living organism). The measurement can indicate the profusion of liquid within the tissue, as well as the oxygenation of the tissue. If an EEG is performed, the controller can receive the outputs from the electrodes. The measurement can represent the electrical activity of the brain of the living organism. A measurement from a motion sensor (e.g., the motion sensor 218 of FIG. 2) can also be received. The measurement can indicate the movement of the living organism.

In step 640, the measurement is transmitted. For example, the controller can transmit the measurement to a computing device (e.g., the computing device 102 of FIGS. 1 & 2). The controller can transmit the measurement via a communication module (e.g., the communications link 112 of FIG. 1 and/or the I/O 204 of FIG. 2). The computing device can determine, based on the measurement, one or more characteristics of the living organism. In an exemplary embodiment, the computing device can determine perfusion and oxygenation information of a brain of the living organism based on the measurement.

In an exemplary embodiment, the measurement transmitted to the computing device indicates the movement of the living organism. The computing device can utilize the movement of the living organism, as well as the measurement form the photodetectors, to filter out any impact that the movement of the living organism may have on the measurements detected from the photodetectors. For example, the motion of the animal can impact the light measurements received by the photodetectors. As an example, the photodetectors can receive a signal of light, and determine a measurement based on the signal of light. However, the detected measurement of light may be different depending on if the animal is still versus if the animal is moving. That is, the movement of the animal can introduce artifacts into the light as measured by the photodetectors. Thus, the motion data can be utilized to filter (e.g., remove) any artifacts that motion of the animal might have introduced into the light as measured by the photodetectors. Therefore, the computing device can utilize the motion data to filter out any artifacts that may have been introduced into the measurement of light by the movement of the animal. Accordingly, the computing device can utilize the motion data to ensure that the light measured by the photodetectors is accurate regardless if the animal is still or moves during the time the measurement is obtained.

In an exemplary embodiment, the controller and/or the computing device can calibrate the photodetectors. For example, the controller and/or the computing device can determine the output for each of the photodetectors when all of the light sources are inactive (e.g., turned off). The controller and/or the computing device can use this information to determine the background light and/or noise detected by the photodetectors so that the background light and/or noise can be filtered out. As another example, the controller and/or the computing device can utilize the background light to calibrate the photodetectors to improve the measurements of the photodetectors. The controller and/or the computing device can also calibrate each of the photodetectors individually because each photodetector may receive different amounts of background light.

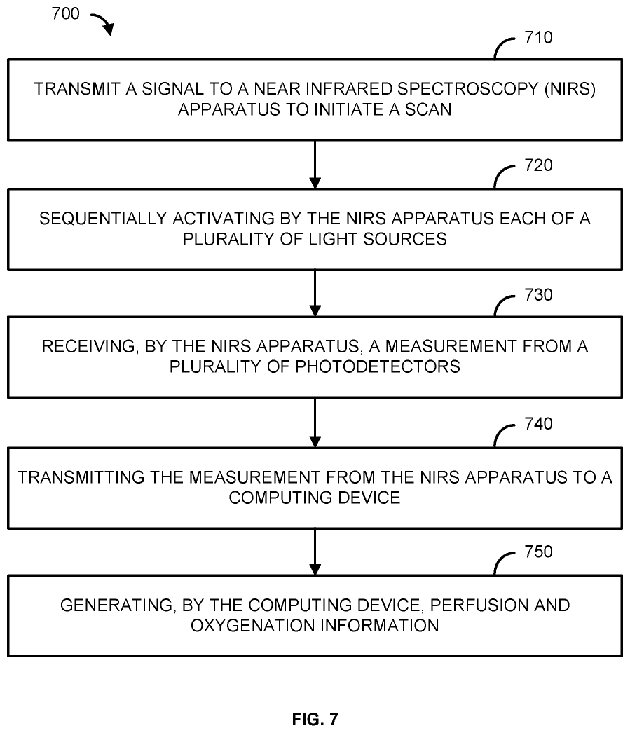

FIG. 7 is a flowchart of an example method 700. At step 710, a signal is transmitted to a Near Infrared Spectroscopy (NIRS) apparatus to initiate a scan. For example, a computing device (e.g., the computing device 102 of FIGS. 1 & 2) transmits a signal to an NIRS apparatus (e.g., the controller 104 of FIGS. 1 & 2 and/or the probe 106 of FIGS. 1-5) to initiate a scan. In one example, the signal to initiate the scan is received via a communications module (e.g., the communications link 112 of FIG. 1 and/or the I/O 204 of FIG. 2).

In step 720, a plurality of light sources can be sequentially activated by the NIRS apparatus. For example, the controller can sequentially activate the light sources (e.g., the light sources 214 of FIG. 2, 302 of FIG. 3, 404 of FIG. 4, and/or 504 of FIG. 5) to emit infrared light. The controller can automatically activate the light sources in response to receiving the signal to initiate a scan. The light sources can output the same wavelength of infrared light or different wavelengths of infrared light. The light sources can be positioned a first distance (e.g., the distance 408 of FIG. 4A) and a second distance (e.g., the distance 410 of FIG. 4A) from a plurality of photodetectors (e.g., the photodetectors 216 of FIG. 2, 304 of FIG. 3, 406 of FIG. 4, and/or 502 of FIG. 5). The light sources can be located on a skull (e.g., the skull 308 of FIG. 3, the animal skull 402 of FIG. 4, and/or the skull 500 of FIG. 5), and the light sources can output light into the tissue (e.g., the tissue 312 of FIG. 3 and/or the brain 506 of FIG. 5) within the skull.

As another example, in step 720 a plurality of electrodes can be activated to perform an EEG. For example, the controller can activate the electrodes (e.g., the electrodes 452 of FIG. 4B). The controller can automatically activate the electrodes in response to receiving the signal to initiate the scan. The electrodes can be located on the skull, and the electrodes can monitor the tissue within the skull. While activating the electrodes is described separately from activating the light sources, a person skilled in the art would appreciate that the plurality of light sources may be activated at the same time as the electrodes. That is, the controller may perform two scans concurrently. One scan using the light sources and photodetectors, and one scan using the electrodes. Further, the two different scans can be performed one after the other such that once the first scan is completed, the second scan automatically begins. However, the scans can also be performed at separate times.

In step 730, a measurement from a plurality of photodetectors is received by the NIRS apparatus. For example, the controller can receive the outputs from the photodetectors. The measurement can represent the detected infrared light (e.g., the light 310 of FIG. 3 and/or the light 508 of FIG. 5) scattered within a living organism (e.g., the animal 108 of FIG. 1). For example, the measurement can represent the detected light scattered within the tissue of the skull of the living organism. The measurement can indicate the perfusion of liquid within the tissue, as well as the oxygenation of the tissue. If an EEG is performed, the controller can receive the outputs from the electrodes (e.g., the electrodes 452 of FIG. 4B). The measurement can represent the electrical activity of the brain of the living organism. A measurement from a motion sensor (e.g., the motion sensor 218 of FIG. 2) can also be received. The measurement can indicate the movement of the living organism.

In step 740, the measurement is transmitted from the NIRS apparatus to a computing device. For example, the controller can transmit the measurement to a computing device (e.g., the computing device 102 of FIG. 4B). The controller can transmit the measurement via a communication module (e.g., the communications link 112 of FIG. 1 and/or the I/O 204 of FIG. 2).

In step 750, perfusion and oxygenation information for the living organism is generated by the computing device. For example, the computing device can perform data analysis on the received signals to determine the perfusion and oxygenation information for the living organism. If an EEG is performed, the measurement can be used to produce a EEG graph that indicates the electrical activity of the brain.

In an exemplary embodiment, the measurement transmitted to the computing device indicates the movement of the living organism. The computing device can utilize the movement of the living organism, as well as the measurement form the photodetectors, to filter out any impact that the movement of the living organism may have on the measurements detected from the photodetectors. For example, the motion of the animal can impact the light measurements received by the photodetectors. As an example, the photodetectors can receive a signal of light, and determine a measurement based on the signal of light. However, the detected measurement of light may be different depending on if the animal is still versus if the animal is moving. That is, the movement of the animal can introduce artifacts into the light as measured by the photodetectors. Thus, the motion data can be utilized to filter (e.g., remove) any artifacts that motion of the animal might have introduced into the light as measured by the photodetectors. Therefore, the computing device can utilize the motion data to filter out any artifacts that may have been introduced into the measurement of light by the movement of the animal. Accordingly, the computing device can utilize the motion data to ensure that the light measured by the photodetectors is accurate regardless if the animal is still or moves during the time the measurement is obtained.

In an exemplary embodiment, the controller and/or the computing device can calibrate the photodetectors. For example, the controller and/or the computing device can determine the output for each of the photodetectors when all of the light sources are inactive (e.g., turned off). The controller and/or the computing device can use this information to determine the background light and/or noise detected by the photodetectors so that the background light and/or noise can be filtered out. As another example, the controller and/or the computing device can utilize the background light to calibrate the photodetectors to improve the measurements of the photodetectors. The controller and/or the computing device can also calibrate each of the photodetectors individually because each photodetector may receive different amounts of background light.

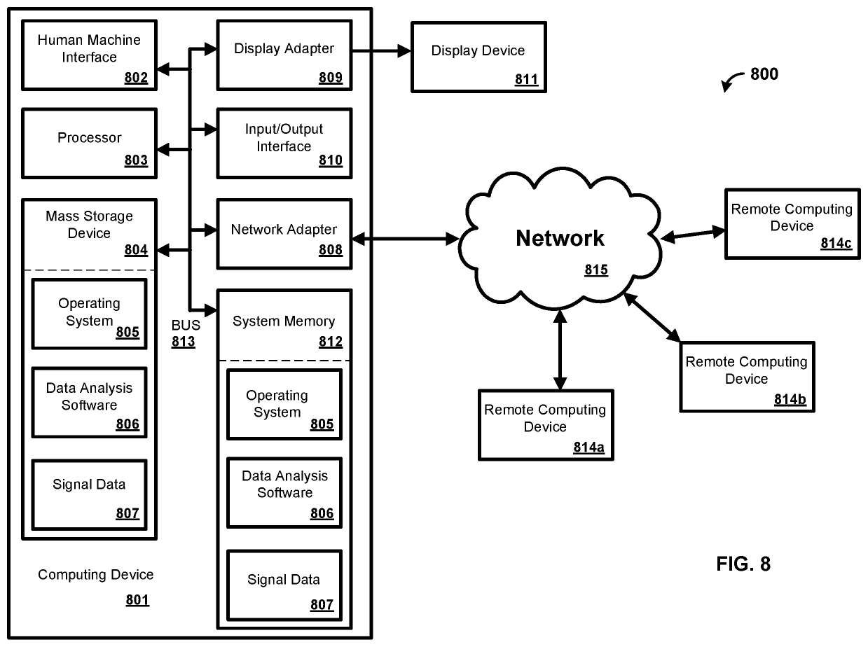

FIG. 8 shows an example of an operating environment 800 including a computing device 801. The computing device 102 of FIGS. 1 & 2, the controller 104 of FIGS. 1 & 2, and the probe 106 of FIGS. 1-5 can include any and all of the functionality of the computing device 801. The operating environment 800 is only an example of an operating environment and is not intended to suggest any limitation as to the scope of use or functionality of operating environment architecture. Neither should the operating environment 800 be interpreted as having any dependency or requirement relating to any one or combination of components illustrated in the operating environment 800.

The present methods and systems can be operational with numerous other general purpose or special purpose computing system environments or configurations. Examples of well-known computing systems, environments, and/or configurations that can be suitable for use with the systems and methods comprise, but are not limited to, personal computers, server computers, laptop devices, and multiprocessor systems. Additional examples comprise set top boxes, programmable consumer electronics, network PCs, minicomputers, mainframe computers, distributed computing environments that comprise any of the above systems or devices, and the like.

The processing of the disclosed methods and systems can be performed by software components. The disclosed systems and methods can be described in the general context of computer-executable instructions, such as program modules, being executed by one or more computers or other devices. Generally, program modules comprise computer code, routines, programs, objects, components, data structures, and/or the like that perform particular tasks or implement particular abstract data types. The disclosed methods can also be practiced in grid-based and distributed computing environments where tasks are performed by remote processing devices that are linked through a communications network. In a distributed computing environment, program modules can be located in local and/or remote computer storage media including memory storage devices.

Further, one skilled in the art will appreciate that the systems and methods disclosed herein can be implemented via a general-purpose computing device in the form of a computing device 801. The computing device 801 can comprise one or more components, such as one or more processors 803, a system memory 812, and a bus 813 that couples various components of the computing device 801 including the one or more processors 803 to the system memory 812. In the case of multiple processors 803, the system can utilize parallel computing.

The bus 813 can comprise one or more of several possible types of bus structures, such as a memory bus, memory controller, a peripheral bus, an accelerated graphics port, and a processor or local bus using any of a variety of bus architectures. By way of example, such architectures can comprise an Industry Standard Architecture (ISA) bus, a Micro Channel Architecture (MCA) bus, an Enhanced ISA (EISA) bus, a Video Electronics Standards Association (VESA) local bus, an Accelerated Graphics Port (AGP) bus, and a Peripheral Component Interconnects (PCI), a PCI-Express bus, a Personal Computer Memory Card Industry Association (PCMCIA), Universal Serial Bus (USB) and the like. The bus 813, and all buses specified in this description can also be implemented over a wired or wireless network connection and one or more of the components of the computing device 801, such as the one or more processors 803, a mass storage device 804, an operating system 805, data analysis software 806, data analysis data 807, a network adapter 808, a system memory 812, an Input/Output Interface 810, a display adapter 809, a display device 811, and a human machine interface 802, can be contained within one or more remote computing devices 814a,b,c at physically separate locations, connected through buses of this form, in effect implementing a fully distributed system.

The computing device 801 typically comprises a variety of computer readable media. As an example, readable media can be any available media that is accessible by the computing device 801 and comprises, for example and not meant to be limiting, both volatile and non-volatile media, removable and non-removable media. The system memory 812 can comprise computer readable media in the form of volatile memory, such as random access memory (RAM), and/or non-volatile memory, such as read only memory (ROM). The system memory 812 typically can comprise data such as signal data 807 and/or program modules such as operating system 805 and data analysis software 806 that are accessible to and/or are operated on by the one or more processors 803.

In another example, the computing device 801 can also comprise other removable/non-removable, volatile/non-volatile computer storage media. The mass storage device 804 can provide non-volatile storage of computer code, computer readable instructions, data structures, program modules, and other data for the computing device 801. For example, a mass storage device 804 can be a hard disk, a removable magnetic disk, a removable optical disk, magnetic cassettes or other magnetic storage devices, flash memory cards, CD-ROM, digital versatile disks (DVD) or other optical storage, random access memories (RAM), read only memories (ROM), electrically erasable programmable read-only memory (EEPROM), and the like.

Optionally, any number of program modules can be stored on the mass storage device 804, including by way of example, an operating system 805 and data analysis software 806. One or more of the operating system 805 and data analysis software 806 (or some combination thereof) can comprise program modules and the data analysis software 806. The signal data 807 can also be stored on the mass storage device 804. The signal data 807 can be stored in any of one or more databases known in the art. Examples of such databases comprise, DB2®, Microsoft® Access, Microsoft® SQL Server, Oracle®, mySQL, PostgreSQL, and the like. The databases can be centralized or distributed across multiple locations within the network 815.

In one example, the data analysis software 806 includes the functionality to operate the controller 104. For example, the data analysis software 806 includes the functionality to communicate with the controller 104 and provide operational instructions and/or preferences to the controller 104. As an example, data analysis software 806 can receive data from the probe 106, and the data analysis software 806 can use the data to determine how the probe 106 should be controlled. The data analysis software 806 can instruct the controller 104 to selectively activate one or more of the light sources of the probe 106. The data analysis software 806 can instruct the controller 104 to automatically activate the light sources and the detectors. For example, the data analysis software 806 can instruct the controller 104 to activate a scan using the probe 106. As another example, the data analysis software 806 can receive input from a user that instructs the data analysis software 806 to have the controller 104 activate a scan using the probe 106.

As another example, the data analysis software 806 can provide settings to the controller 104 that indicate when the controller 104 should activate the light source 214 in order to measure signals. As one example, the data analysis software 806 can provide start and stop times that the controller 104 should activate the light source 214. As another example, the data analysis software 806 can indicate times that the controller 104 should start dynamically managing the probe 106. As a further example, the data analysis software 806 can provide settings as to when the controller 104 should perform a scan using the probe 106. In one example, a user of the data analysis software 806 actively selects the instructions or settings that are transmitted to the controller 104. In another example, the data analysis software 806 dynamically decides the instructions or settings that are transmitted to the controller 104 without input from a user. In another example, the data analysis software 806 receives input from a user indicating the preferences and/or settings the user would like the data analysis software 806 to implement. The data analysis software 806 can then automatically transmit instructions to the controller 104 based on the user indicated preferences and/or settings. In one example, the user of the data analysis software 806 selects specific setting related to a scan using the probe 106.

In one example, the data analysis software 806 can run data analysis on the signals output from the probe 106. For example, the probe 106 can provide instantaneous output signals. The data analysis software 806 can store the output signals from the probe 106 and convert the output signals into a data.

In one example, the data analysis software 806 is a web based or telecommunications based server that has an associated interface that a user can access which controls the functionality of the controller 104 and the probe 106.

In another example, the user can enter commands and information into the computing device 801 via an input device (not shown). Examples of such input devices comprise, but are not limited to, a keyboard, pointing device (e.g., a computer mouse, remote control), a microphone, a joystick, a scanner, tactile input devices such as gloves, and other body coverings, motion sensor, and the like. These and other input devices can be connected to the one or more processors 803 via a human machine interface 802 that is coupled to the bus 813, but can be connected by other interface and bus structures, such as a parallel port, game port, an IEEE 1394 Port (also known as a Firewire port), a serial port, network adapter 808, and/or a universal serial bus (USB).

In yet another example, a display device 811 can also be connected to the bus 813 via an interface, such as a display adapter 809. It is contemplated that the computing device 801 can have more than one display adapter 809 and the computing device 801 can have more than one display device 811. For example, a display device 811 can be a monitor, an LCD (Liquid Crystal Display), light emitting diode (LED) display, television, smart lens, smart glass, and/or a projector. In addition to the display device 811, other output peripheral devices can comprise components such as speakers (not shown) and a printer (not shown) which can be connected to the computing device 801 via Input/Output Interface 810. Any step and/or result of the methods can be output in any form to an output device. Such output can be any form of visual representation, including, but not limited to, textual, graphical, animation, audio, tactile, and the like. The display 811 and the computing device 801 can be part of one device, or separate devices.

The computing device 801 can operate in a networked environment using logical connections to one or more remote computing devices 814a,b,c. By way of example, a remote computing device 814a,b,c can be a personal computer, computing station (e.g., workstation), portable computer (e.g., laptop, mobile phone, tablet device), smart device (e.g., smartphone, smart watch, activity tracker, smart apparel, smart accessory), security and/or monitoring device, a server, a router, a network computer, a peer device, edge device or other common network node, and so on. As an example, remote computing devices 814a,b,c can be the computing device 102, the controller 104, and the probe 106. Logical connections between the computing device 801 and a remote computing device 814a,b,c can be made via a network 815, such as a local area network (LAN) and/or a general wide area network (WAN). Such network connections can be through a network adapter 808. A network adapter 808 can be implemented in both wired and wireless environments. Such networking environments are conventional and commonplace in dwellings, offices, enterprise-wide computer networks, intranets, and the Internet.

For purposes of illustration, application programs and other executable program components such as the operating system 805 are shown herein as discrete blocks, although it is recognized that such programs and components can reside at various times in different storage components of the computing device 801, and are executed by the one or more processors 803 of the computing device 801. An implementation of data analysis software 806 can be stored on or transmitted across some form of computer readable media. Any of the disclosed methods can be performed by computer readable instructions embodied on computer readable media. Computer readable media can be any available media that can be accessed by a computer. By way of example and not meant to be limiting, computer readable media can comprise “computer storage media” and “communications media.” “Computer storage media” can comprise volatile and non-volatile, removable and non-removable media implemented in any methods or technology for storage of information such as computer readable instructions, data structures, program modules, or other data. Exemplary computer storage media can comprise RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by a computer.

The methods and systems can employ artificial intelligence (AI) techniques such as machine learning and iterative learning. Examples of such techniques include, but are not limited to, expert systems, case based reasoning, Bayesian networks, behavior based AI, neural networks, fuzzy systems, evolutionary computation (e.g. genetic algorithms), swarm intelligence (e.g. ant algorithms), and hybrid intelligent systems (e.g. Expert inference rules generated through a neural network or production rules from statistical learning).

While the methods and systems have been described in connection with specific examples, it is not intended that the scope be limited to the particular examples set forth, as the examples herein are intended in all respects to be possible examples rather than restrictive.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including: matters of logic with respect to arrangement of steps or operational flow; plain meaning derived from grammatical organization or punctuation; the number or type of examples described in the specification.

It will be apparent to those skilled in the art that various modifications and variations can be made without departing from the scope or spirit. Other examples will be apparent to those skilled in the art from consideration of the specification and practice disclosed herein. It is intended that the specification and examples be considered as exemplary only, with a true scope and spirit being indicated by the following claims.

What is claimed is:

1. A system, comprising:
a probe comprising a plurality of light sources and a plurality of photodetectors,
wherein the plurality of light sources are positioned a first distance from a first portion of the plurality of photodetectors and a second distance from a second portion of the plurality of photodetectors,
wherein the plurality of light sources are configured to emit light,
wherein the plurality of photodetectors are configured to detect the light scattered in a living organism; and
a controller comprising a communications module, wherein the controller is in communication with the probe, and wherein the controller is configured to,
receive, via the communications module, a signal from a computing device to initiate a scan;
responsive to the signal to initiate the scan, sequentially activate each of the plurality of light sources to emit light,
receive, based on the sequential activation, a measurement from the plurality of photodetectors, wherein the measurement represents detected light scattered in the living organism, and
transmit, via the communications module to the computing device, the measurement.

2. The system of claim 1, wherein the probe further comprises a plurality of electrodes, and wherein the controller is further configured to perform an electroencephalography (EEG) scan using the electrodes.

3. The system of claim 1, further comprising:
a stable current source for the plurality of light sources;
a battery; and
a voltage regulator configured to provide a constant voltage.

4. The system of claim 1, wherein the controller is further configured to receive a measurement of a background light level at each of the plurality of photodetectors while all of the plurality of light sources are inactive.

5. The system of claim 4, wherein the controller is further configured to calibrate each of the plurality of photodetectors based on the background light level.

6. The system of claim 1, further comprising a multiplexer configured to:
receive the outputs from the plurality of photodetectors;
amplify the received outputs;
filter the received outputs; and
digitize the received outputs.

7. The system of claim 5, wherein the digitized outputs represent spectral information characterizing detected light scattered in the living organism.

8. The system of claim 1, wherein the plurality of light sources comprise a plurality of Light Emitting Diode (LEDs), and wherein the plurality of photodetectors comprise a plurality of photodiodes.

9. The system of claim 7, wherein the plurality of photodiodes comprises six or eight photodiodes, wherein each photodiode comprises six optical channels configured for monitoring bilateral motor and somatosensory cortices of the living organism.

10. The system of claim 1, wherein the plurality of light sources and the plurality of photodetectors are mounted on a flexible film.

11. The system of claim 1, wherein the first portion of the plurality of photodetectors are configured to sample light absorption changes in a short pathway through superficial tissues of the living organism.

12. The system of claim 1, wherein the second portion of the plurality of photodetectors are configured to sample light absorption changes in a long pathway through deep tissues of the living organism.

13. The system of claim 1, wherein the light comprises infrared light and red light, and wherein the second distance is different from the first distance.

14. The system of claim 1, wherein the probe further comprises a motion sensor configured to detect motion of the living organism.

15. A method, comprising:
receiving, via a communications module from a computing device, a signal to initiate a scan;
responsive to receiving the signal to initiate the scan, sequentially activating each of a plurality of light sources to emit light,
wherein the plurality of light sources are positioned a first distance from a first portion of a plurality of photodetectors and a second distance from a second portion of the plurality of photodetectors;
receiving, based on the sequential activation, a measurement from the plurality of photodetectors, wherein the measurement represents detected light scattered in a living organism; and
transmitting, via the communications module to the computing device, the measurement.

16. The method of claim 14, further comprising receiving a measurement of a background light level at each of the plurality of photodetectors while all of the plurality of light sources are inactive, and calibrating each of the plurality of photodetectors based on the measurement of the background light.

17. The method of claim 14, wherein the plurality of light sources comprise a plurality of Light Emitting Diodes (LEDs), and wherein the plurality of photodetectors comprise a plurality of photodiodes.

18. A method, comprising:
wirelessly transmitting, from a computing device to a Near Infrared Spectroscopy (NIRS) apparatus, a signal to initiate a scan;
responsive to the signal to initiate the scan, sequentially activating each of a plurality of light sources of the NIRS apparatus to emit infrared light,
wherein the plurality of light sources are positioned a first distance from a first portion of a plurality of photodetectors and a second distance from a second portion of the plurality of photodetectors;
receiving, based on the sequential activation, a measurement from the plurality of photodetectors, the measurement representing detected infrared light scattered in a living organism;
transmitting, from the NIRS apparatus to the computing device, the measurement; and
generating, by the computing device based on the measurement, perfusion and oxygenation information for the living organism.

19. The method of claim 17, wherein the plurality of light sources comprise a plurality of Light Emitting Diodes (LEDs), and wherein the plurality of photodetectors comprise a plurality of photodiodes.

20. The method of claim 17, wherein each of the plurality of light sources further emit red light.
